(19) United States
(12) Patent Application Publication
(10) Pub. No.: US 2026/0215860 A1
(43) Pub. Date: Jul. 30, 2026

(54) ROBOTIC CATHETER MODULE FOR TRANSLATION AND ROTATION OF AN ELONGATE FLEXIBLE MEDICAL INSTRUMENT

(71) Applicant: ROBOCATH, Rouen (FR)

(72) Inventors: Wilfried Poirier, Sotteville-lès-Rouen (FR); Sébastien Deboeuf, Bonsecours (FR)

(73) Assignee: ROBOCATH, Rouen (FR)

(21) Appl. No.: 19/141,461

(22) PCT Filed: Dec. 19, 2023
(86) PCT No.: PCT/EP2023/086657

§ 371 Date: Jun. 20, 2025

(30) Foreign Application Priority Data
Dec. 23, 2022 (FR) FR2214373

(51) Int. Cl. A61B 34/30

(52) U.S. Cl. CPC A61B 34/30; A61B 2034/301

(57) ABSTRACT

A robotic catheter module for translation and rotation of an elongate flexible medical instrument, including: a support (S); at least one pair of movable pads arranged face to face and adapted to be able to effect a longitudinal translation of the elongate flexible medical instrument and to effect a rotation of the elongate flexible medical instrument about the longitudinal direction; and a device for driving the pads, including three drive members for driving the pads in three different directions (y, z, x), each with an actuator which is connected to the pads via at least one rolling-bearing slide connection, these three drive members for the pads being independent of one another, such that the actuation of an actuator of one of the drive members does not displace the actuators of the two other drive members.

### FIELD

The invention relates to the technical field of robotic catheter modules for translation and rotation of an elongate flexible medical instrument. The elongate flexible medical instrument may be, in particular, a catheter, a catheter guide, or a guide catheter.

### BACKGROUND

A prior art document, described in Patent Application WO 2016/198800, describes transmission of the pad-holder movement that is carried out in the following manner: for each of the three axes x, y and z, a plate makes the connection between the pad-holder and a linear actuator. Each plate comprises an opening in its center to enable the movements of other plates, and thus to enable a completely independent movement for the pad-holder on each of the three axes x, y, and z.

However, this technical solution presents a connection between the plates that leads to plane-to-plane friction due to one plate sliding on the two other plates. This friction reduces the precision of the system and its responsiveness, and tends to reduce its service life in the long term.

### SUMMARY

The goal of the present invention is to provide a robotic catheter module for translation and rotation of an elongate flexible medical instrument, at least partially mitigating the above-mentioned disadvantages.

More specifically, the invention aims to provide a robotic catheter module for translation and rotation of an elongate flexible medical instrument that presents improvements concerning the precision of the system, its responsiveness, and its service life. In this robotic catheter module for translation and rotation of an elongate flexible medical instrument, the existing friction has been reduced, while the independence and the efficacy in actuating the movements of the pad-holder(s) along the three axes x, y, and z, has been retained.

To do this, in the robotic catheter module for translation and rotation of an elongate flexible medical instrument, the slide connections have been replaced by rolling-bearing connections at particular locations in the mechanism of the robotic catheter module for translation and rotation of an elongate flexible medical instrument, in order to reduce friction in the mechanism during operation.

To this effect, the present invention proposes a robotic catheter module for translation and rotation of an elongate flexible medical instrument, comprising: a support having a longitudinal direction, a transverse direction which is orthogonal to the longitudinal direction, and a vertical direction which is orthogonal to the longitudinal direction and to the transverse direction; at least one pair of movable pads arranged face to face, adapted to be able to: effect a longitudinal translation of the elongate flexible medical instrument, by gripping the elongate flexible medical instrument between the pads and by moving the pads together in translation in the longitudinal direction; effect a rotation of the elongate flexible medical instrument about the longitudinal direction, by gripping the elongate flexible medical instrument between the pads and by moving the pads together in translation in the vertical direction, said pads moving in translation in opposite directions; a device for driving pads, comprising: a first drive member for driving the pads in the longitudinal direction (y), comprising a first actuator which is connected to the pads via at least one rolling-bearing slide connection; a second drive member for driving the pads in the vertical direction (z), comprising a second actuator which is connected to the pads via at least one rolling-bearing slide connection; a third drive member for driving the pads at least relative to one another in the transverse direction (x), comprising a third actuator which is connected to at least one pad via at least one rolling-bearing slide connection; these three drive members for the pads being independent of one another such that the actuation of an actuator of one of the drive members does not displace the actuators of the two other drive members.

To this effect, the present invention also proposes a robotic catheter module for the translation and/or rotation of an elongate flexible medical instrument, comprising: a support having a longitudinal direction, a transverse direction which is orthogonal to the longitudinal direction, and a vertical direction which is orthogonal to the longitudinal direction and to the transverse direction; at least one pair of movable pads arranged face to face, adapted to be able to: effect a translation in the longitudinal direction of the elongate flexible medical instrument, by a first translation cycle: by gripping the elongate flexible medical instrument between the pads, moving the pads together in translation longitudinally in one direction, releasing the elongate flexible medical instrument, moving the pads together in translation in the opposite direction longitudinally; effect a rotation of the elongate flexible medical instrument about the longitudinal direction, by a second rotation cycle: by gripping the elongate flexible medical instrument between the pads, moving the pads together in translation vertically but in opposite directions, releasing the elongate flexible medical instrument, moving the pads in translation vertically in the opposite direction; a device for driving the movable pads comprising: a first drive member for driving pads in the longitudinal direction, comprising a first actuator which is connected to the pads via at least one rolling-bearing slide connection; a second drive member for driving pads in the vertical direction, comprising a second actuator which is connected to the pads via at least one rolling-bearing slide connection; a third drive member for driving pads at least relative to one another in the transverse direction, comprising a third actuator which is connected to at least one pad via at least one rolling-bearing slide connection; these three drive members for the pads being independent of one another such that the actuation of an actuator of one of the drive members does not displace the actuators of the two other drive members.

Another improvement has also been achieved, which may be used either cumulatively or alternatively to the previous improvement. This other improvement consists of placing a sliding ball joint, to allow both an axial misalignment and a radial misalignment between two shafts. When the sliding ball joint is combined with a rolling-bearing connection or rolling-bearing connections, the obtained system is optimal because it is effective and fluid, while remaining fairly tolerant concerning manufacturing deviations and the positioning of the various parts forming the mechanism of the robotic catheter module for translation and rotation of an elongate flexible medical instrument.

To this effect, the invention further proposes a robotic catheter module for translation and/or rotation of an elongate flexible medical instrument, characterized in that said module comprises a sliding ball joint adjustable for radial misalignment and adjustable for axial misalignment, mounted about a shaft so as to slide radially about said shaft.

According to preferred embodiments, the invention comprises one or more of the following characteristics which may be used separately or in a combination of some of them or all of them with one or more of the aforementioned objects of the invention.

Preferably, one among the first drive member, the second drive member, and the third drive member comprises a sliding coupling that is connected to the actuator of said drive member by a first shaft and that is connected to at least one pad by a second shaft, the sliding coupling allowing a translational movement of the second shaft relative to the first shaft along a plane perpendicular to the first shaft.

Thus, the independence of the displacements along the three axes x, y, and z of the pad-holder(s) is maintained through a simple and effective mechanism that occupies less space.

The different possible combinations are:
Only the first drive member comprises such a sliding coupling,
Only the second drive member comprises such a sliding coupling,
Only the third drive member comprises such a sliding coupling,
Only the first drive member and second drive member each comprise such a sliding coupling,
Only the second drive member and third drive member each comprise such a sliding coupling,
Only the third drive member and first drive member each comprise such a sliding coupling,
The first drive member and second drive member and third drive member each comprise such a sliding coupling.

Preferably, the sliding coupling via rolling bearing comprises several balls, said balls being respectively housed in several housings supported by a same support, and able to roll on a same plane of a same part.

Thus, the use of balls in housings presents a good compromise between the fluidity of the connection made by the sliding coupling, on the one hand, and, on the other hand, the stability and robustness of this sliding coupling.

Preferably, the coupling comprises at least three balls.

Thus, the stability of the sliding coupling is optimal.

Preferably, the sliding coupling is installed on the output shaft of the third drive member.

A good compromise between fluidity of the connection and stability of the sliding coupling is thus obtained for the displacement along the x axis for gripping the elongate flexible medical instrument between pads supported by pad-holders, the transverse x axis being particularly sensitive to discrepancies and misalignments.

Preferably, the second actuator is a linear actuator and the second drive member comprises a conversion device that converts a translational movement of the second actuator into a translational movement of the pads along the vertical axis by means of an intermediate adapter.

A good compromise is thus made between simplicity of the actuator used, and the reduced bulkiness of the entire mechanism due to the parallel orientation of the second and third actuators.

Preferably, the intermediate adapter has an L shape and the axis of rotation of said intermediate adapter is located at the intersection of the two arms of the L, a first arm of the L being connected to a pad, the second drive member being connected to a second arm of the L, the first arm of the L preferably being shorter than the second arm of the L or else the first arm of the L preferably being at least 2 times or at least 3 times shorter than the second arm of the L.

The adapter thus retains a simple and robust shape, while carrying out sophisticated kinematics. This is possible, in particular, since the amplitude of displacement of the pad-holders along the vertical axis z remains very limited.

Preferably, the intermediate adapter comprises a sliding pivot connection at each of its two ends.

The adapter thus retains a simple and robust shape, while carrying out sophisticated kinematics. This is possible, in particular, since the amplitude of displacement of the pad-holders along the vertical axis z remains very limited.

Preferably, at least one among the first drive member, the second drive member, and the third drive member comprises a sliding ball joint for adjusting radial misalignment and for adjusting axial misalignment, mounted about an output shaft so as to slide radially about said shaft.

Thus, when the sliding ball joint is combined with a rolling-bearing connection or rolling-bearing connections, the obtained system is optimal because it is effective and fluid, while remaining fairly tolerant concerning manufacturing deviations and the positioning of the various parts forming the mechanism of the robotic catheter module for translation and rotation of an elongate flexible medical instrument.

The different possible combinations are:
Only the first drive member comprises such a sliding ball joint,
Only the second drive member comprises such a sliding ball joint,
Only the third drive member comprises such a sliding ball joint,
Only the first drive member and second drive member each comprise such a sliding ball joint,
Only the second drive member and third drive member each comprise such a sliding ball joint,
Only the third drive member and first drive member each comprise such a sliding ball joint,
The first drive member and second drive member and third drive member each comprise such a sliding ball joint.

Preferably, the first drive member comprises a single first actuator, the second drive member comprises a single second actuator, and the third drive member comprises a single third actuator.

The overall bulkiness of the mechanism is thus greatly reduced.

Other features and advantages of the invention will become apparent upon reading the following description of a preferred embodiment of the invention, given by way of example and with reference to the appended drawings.

### BRIEF DESCRIPTION OF THE DRAWINGS

FIG. 1 schematically represents a perspective view of an example of a robotic platform integrating the inner mechanism of a robotic catheter module for translation and rotation of an elongate flexible medical instrument, according to one embodiment of the invention.

FIG. 2 schematically represents a front view of the same example of a robotic platform integrating the inner mechanism of a robotic catheter module for translation and rotation of an elongate flexible medical instrument, according to one embodiment of the invention.

FIG. 3 schematically represents a top view of the same example of a robotic platform integrating the inner mechanism of a robotic catheter module for translation and rotation of an elongate flexible medical instrument, according to one embodiment of the invention.

FIG. 4 schematically represents a perspective view of a linear actuator, along the x axis of the same example of a robotic platform integrating the inner mechanism of a robotic catheter module for translation and rotation of an elongate flexible medical instrument, according to one embodiment of the invention.

FIG. 5 schematically represents a perspective view of a sliding ball joint from the same example of a robotic platform integrating the inner mechanism of a robotic catheter module for translation and rotation of an elongate flexible medical instrument, according to one embodiment of the invention.

FIG. 6 schematically represents a front view of a sliding ball joint from the same example of a robotic platform integrating the inner mechanism of a robotic catheter module for translation and rotation of an elongate flexible medical instrument, according to an embodiment of the invention.

FIG. 7 schematically represents a cross-sectional view of a sliding ball joint from the same example of a robotic platform integrating the inner mechanism of a robotic catheter module for translation and rotation of an elongate flexible medical instrument, according to one embodiment of the invention.

FIG. 8 schematically represents a first relative position between two shafts in a robotic platform.

FIG. 9 schematically represents a first configuration of a sliding ball joint corresponding to the first relative position between two shafts in a robotic platform.

FIG. 10 schematically represents a second relative position between two shafts in a robotic platform.

FIG. 11 schematically represents a second configuration of a sliding ball joint corresponding to the second relative position between two shafts in a robotic platform.

FIG. 12 schematically represents a third relative position between two shafts in a robotic platform.

FIG. 13 schematically represents a third configuration of a sliding ball joint corresponding to the third relative position between two shafts in a robotic platform.

FIG. 14 schematically represents a fourth configuration of a sliding ball joint corresponding to a combination of the second and third relative positions between two shafts in a robotic platform.

### DETAILED DESCRIPTION

In all of the following text, the terms “elongate flexible medical instrument” and “medical instrument” will be used interchangeably.

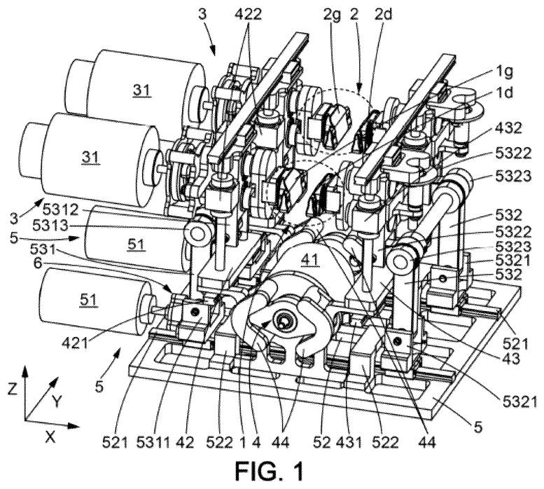

FIG. 1 schematically represents a perspective view of an example of a robotic platform integrating the inner mechanism of a robotic catheter module for translation and rotation of an elongate flexible medical instrument, according to one embodiment of the invention.

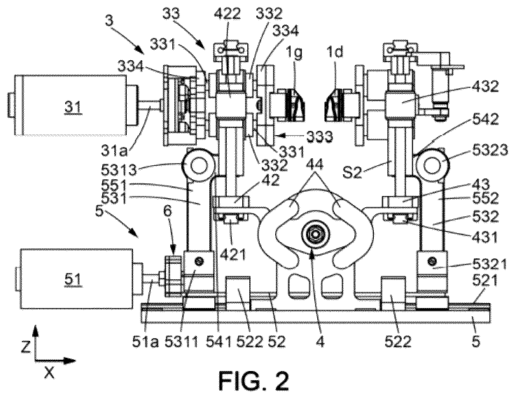

FIG. 2 schematically represents a front view of the same example of a robotic platform integrating the inner mechanism of a robotic catheter module for translation and rotation of an elongate flexible medical instrument, according to one embodiment of the invention.

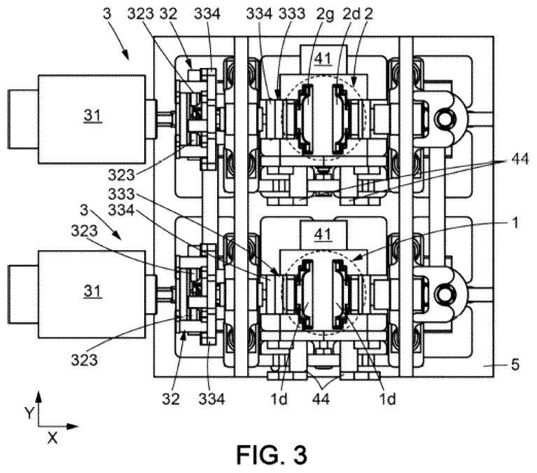

FIG. 3 schematically represents a top view of the same example of a robotic platform integrating the inner mechanism of a robotic catheter module for translation and rotation of an elongate flexible medical instrument, according to one embodiment of the invention.

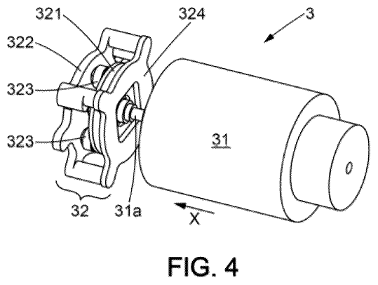

FIG. 4 schematically represents a perspective view of a linear actuator along the x axis of the same example of a robotic platform integrating the inner mechanism of a robotic catheter module for translation and rotation of an elongate flexible medical instrument, according to one embodiment of the invention.

The operation of the robotic platform integrating the inner mechanism of a robotic catheter module for translation and rotation of an elongate flexible medical instrument will now be explained in connection with FIGS. 1 to 4. The robotic platform is supported by a support S.

The robotic platform comprises a first pair 1 of pad-holders, and a second pair 2 of pad-holders. First pair 1 comprises pad-holders 1g and 1d which are arranged face to face, and second pair 2 comprises pad-holders 2g and 2d which are arranged face to face. Pad-holders 1g and 2g are arranged on one side of the robotic platform (on the left in FIGS. 1 to 4), while pad-holders 1d and 2d are arranged on the other side of the robotic platform (on the right in FIGS. 1 to 4).

Pad-holders 1g, 1d, 2g, 2d are each intended to receive one pad by means of which said pad-holders drive the medical instrument. The fixing of a pad on a pad-holder 1g, 1d, 2g, 2d is described in document WO2015189529 (incorporated with reference to the present patent application). Each pair of pad-holders reproduces the movement of the thumb and index finger of a practitioner manipulating the medical instrument.

In FIGS. 1 to 4, the robotic platform represented comprises two pairs of pad-holders. However, the number of pairs of pad-holders may vary. Thus, the robotic platform may comprise a single pair of pad-holders, or else a number greater than two pairs of pad-holders. A higher number of pairs of pad-holders may be used to manipulate several medical instruments simultaneously, the example of a platform represented in FIGS. 1 to 4 being adapted to manipulate a single medical instrument by imparting to it a continuous translational movement and a continuous rotational movement.

As seen in FIGS. 1 to 4, pad-holders 1g and 2g are movable along the x axis, the y axis and the z axis, while pad-holders 1d and 2d are movable only along the y axis and the z axis. The movement of pad-holders along the x axis makes it possible to grip or release the medical instrument. When pad-holders 1g and 1d, respectively covered by their corresponding pads, draw closer to one another by translation along the x axis, they grip the medical instrument between them. When pad-holders 1g and 1d, respectively covered by their corresponding pads, move apart from one another by translation along the x axis, they release the medical instrument from their grip. When pad-holders 2g and 2d, respectively covered by their corresponding pads, draw closer to one another by translation along the x axis, they grip the medical instrument between them. When pad-holders 2g and 2d, respectively covered by their corresponding pads, move apart from one another by translation along the x axis, they release the medical instrument from their grip.

The movement of pad-holders along the y axis makes it possible to impart a translational movement to the medical instrument along its main axis of elongation. When pad-holders 1g and 1d, respectively covered by their corresponding pads, advance simultaneously, by translation along the y axis, while gripping the medical instrument between them, they cause the medical instrument to advance in translation. When pad-holders 1g and 1d, respectively covered by their corresponding pads, move backward simultaneously, by reverse translation along the y axis, while gripping the medical instrument between them, they cause the medical instrument to move backward in translation. When pad-holders 1g and 1d, respectively covered by their corresponding pads, move backward simultaneously by reverse translational movement along the y axis, without gripping the medical instrument between them, they are repositioned so that they are once again able to cause the medical instrument to advance in translation. When pad-holders 1g and 1d, respectively covered by their corresponding pads, advance simultaneously by reverse translational movement along the y axis, without gripping the medical instrument between them, they are repositioned so that they are once again able to cause the medical instrument to move backward in translation. When pad-holders 2g and 2d, respectively covered by their corresponding pads, advance simultaneously by translational movement along the y axis, while gripping the medical instrument between them, they cause the medical instrument to advance in translation. When pad-holders 2g and 2d, respectively covered by their corresponding pads, move backward simultaneously by reverse translational movement along the y axis, while gripping the medical instrument between them, they cause the medical instrument to move backward in translation. When pad-holders 2g and 2d, respectively covered by their corresponding pads, move backward simultaneously by reverse translational movement along the y axis, without gripping the medical instrument between them, they are repositioned so that they are once again able to cause the medical instrument to advance in translation. When pad-holders 2g and 2d, respectively covered by their corresponding pads, advance simultaneously by reverse translational movement along the y axis, without gripping the medical instrument between them, they are repositioned so that they are once again able to cause the medical instrument to move backward in translation. The first pair of pad-holders 1g and 1d on the one hand, and the second pair of pad-holders 2g and 2d on the other hand, operate alternately, i.e. during the time when the first pair of pad-holders 1g and Id are gripping the medical instrument, the second pair of pad-holders 2g and 2d release the medical instrument, and vice versa, so as to cause the medical instrument to advance or move backward in a more fluid manner.

The movement of pad-holders along the z axis makes it possible to impart a rotational movement to the medical instrument about its main axis of elongation, i.e. the y axis when the medical instrument is installed in the robotic platform. When pad-holders 1g and 1d, respectively covered by their corresponding pads, are displaced vertically in phase opposition, i.e. while pad-holder 1g is rising, pad-holder 1d is descending, and vice versa, the medical instrument rotates about itself in one rotational direction or in the opposite rotational direction. When pad-holders 2g and 2d, respectively covered by their corresponding pads, are displaced vertically in phase opposition, i.e. while pad-holder 2g is rising, pad-holder 2d is descending, and vice versa, the medical instrument rotates about itself in one rotational direction or in the opposite rotational direction.

The displacement of pad-holders 1d and 2d along the x axis for gripping is eliminated in order to simplify the robotic platform and reduce its bulkiness. In fact, it is sufficient for only one of the two pad-holders to advance towards the other in order to grip the medical instrument between the two pad-holders of a pair.

The various movements of pad-holders along the x, y, and/or z axis may be either carried out separately, or may be combined two by two, particularly between the movements along the y axis and along the z axis simultaneously.

First, the movement of the two pairs of pad-holders along the x axis alone (for greater clarity) will now be explained.

Movement along the x axis of pad-holders 1g and 2g is ensured by two first drive units 3, a first drive unit 3 being associated with each of pad-holders 1g and 2g. Each of first drive units 3 comprises a linear motor 31 that is connected to a sliding coupling 32 by means of an input shaft 31a, the sliding coupling being connected to pad-holder 1g or 2g via a transmission module 33.

Sliding coupling 32 is a connection which makes it possible to transmit a translation along the x axis, but also enabling translational movements of pad-holder 1g or 2g relative to linear motor 31 along axes y and z. Sliding coupling 32 comprises a first plate 321 connected to linear motor 31, a second plate 322 connected to pad-holder 1g or 2g and arranged facing first plate 321, three balls 323 arranged between first plate 321 and second plate 322, and a stop 324 that holds first plate 321 and second plate 322 tightly around balls 323. In addition, stop 324 allows a displacement of first plate 321 relative to second plate 322 in translational movements along axes y and z. The balls allow the movement between first plate 321 and second plate 322 to take place by rolling and not by sliding, thereby greatly reducing friction.

The function of transmission module 33 is to transmit translational movement along the x axis to the pad-holder to which said transmission module 33 is connected. Transmission module 33 thus comprises at least one output shaft 331 (here two output shafts 331 in the example embodiment illustrated in FIGS. 1 to 4), of which a first end is fixed to sliding coupling 32 and a second end is fixed to pad-holder 1g or 2g. Each output shaft 331 slides in a rolling-bearing slide 332 (i.e. a ball slide or else a needle slide), thereby limiting friction. Slides 332 are fixed on a support S1, support S1 making it possible to transmit translational movements along the y and z axes to pad-holders 1g and 2g via output shafts 331.

Transmission module 33 also comprises a two-way stop 333 that limits the movements of pad-holder 1g or 2g along the x axis in both directions. Advantageously, two-way stop 333 comprises at least one plug 334 made of an elastomer (preferably two plugs 334, one plug 334 for forming a stop in each direction) to soften impacts in order to preserve the mechanism and to reduce noise when two-way stop 333 comes into abutment. Plug 334 blocks translational movement in one direction by abutting against slide 332. In the example illustrated in FIGS. 1 to 4, the two plugs 334 are arranged one on either side of slide 332, for each output shaft 331.

Support 52 remains immobile along the x axis, with no displacement along the x axis.

Second, the movement of the two pairs of pad-holders along the y axis alone (for greater clarity) will now be explained.

Movement along the y axis of pad-holders 1g, 1d, 2g and 2d is ensured by two second drive units 4, a second drive unit 4 being associated with each of the pairs of pad-holders 1 and 2. Each of second drive units 4 comprises a linear motor 41 that is connected, on the one hand, to a first carriage 42 that is movable in translation along a rail 421 oriented along the y axis, and, on the other hand, to a second carriage 43 that is movable in translation along a rail 431 oriented along the y axis. First carriage 42 is connected to support S1 via a slide 422 of axis z. Slide 422 enables first carriage 42 to drive support S1 in translation along the y axis while allowing z-axis translational movements of said support S1. Second carriage 43 is connected to support S2 via a z-axis slide 432. Slide 432 enables second carriage 43 to drive support S2 in translation along the y axis while allowing z-axis translational movements of said support S2.

Advantageously, the movement of first carriage 42 along rail 421 is achieved by rolling and not by sliding, thereby making it possible to limit friction. First carriage 42 is thus mounted on rail 421 with a rolling-bearing slide.

Advantageously, the movement of second carriage 43 along rail 431 is achieved by rolling and not by sliding, thereby making it possible to limit friction. Second carriage 43 is thus mounted on rail 431 with a rolling-bearing slide.

Each drive unit 4 comprises a two-way stop 44 that limits the travel of linear motors 41 along the y axis in the two directions. Advantageously, two-way stop 44 comprises at least one plug made of an elastomer (preferably two plugs, one plug for forming a stop in each direction) to soften impacts in order to preserve the mechanism and to reduce noise when linear motor 41 comes into abutment against two-way stop 44. In the example embodiment illustrated in FIGS. 1 to 4, two-way stops 44 are made as two distinct parts, but two-way stops 44 may be made so as to form a single piece.

Third, the movement of the two pairs of pad-holders along the z axis alone (for greater clarity) will now be explained.

Movement along the z axis of pad-holders 1g, 1d, 2g and 2d is ensured by two third drive units 5, a third drive unit 5 being associated with each of the pairs of pad-holders 1 and 2. Each of the third drive units 5 comprises a linear motor 51 that is connected by means of an input shaft 51 to a third carriage 52 that is movable in translation along a rail 521 oriented along the x axis. Third carriage 52 is connected to a first lever 531 having an L shape and to a second lever 532, also having an L shape. The L of first lever 531 comprises a first arm 541 and a second arm 551, first arm 541 being shorter than second arm 551. The L of second lever 532 comprises a first arm 542 and a second arm 552, first arm 542 being shorter than second arm 552. First lever 531 is fixed at its first end to third carriage 52 via a first sliding pivot 5311 having axis of rotation y and axis of translation y, and is fixed at its second end to support S1 by a second sliding pivot 5312 having axis of rotation y and axis of translation y. Second lever 532 is fixed at its first end to third carriage 52 via a third sliding pivot 5321 having axis of rotation y and axis of translation y, and is fixed at its second end to support S2 by a fourth sliding pivot 5322 having axis of rotation y and axis of translation y. The freedom in translation provided by first sliding pivot 5311, second sliding pivot 5312, third sliding pivot 5321, and fourth sliding pivot 5322 enables first lever 531 and second lever 532 to follow the translational movements along the y axis of pad-holders 1g, 1d, 2g and 2d, transmitted by the two second drive units 4.

Two plugs 522 are positioned on rail 521 so as to form stops and limit the translational movements of third carriage 52 along the x axis in both directions. Advantageously, plugs 522 are made of an elastomer so as to soften impacts in order to preserve the mechanism and to reduce noise when third drive units 5 come into abutment.

Advantageously, the movement of third carriage 52 along rail 521 is achieved by rolling and not by sliding, thereby making it possible to limit friction. Third carriage 52 is thus mounted on rail 521 with a rolling-bearing slide.

Conversion of translational movement along the x axis of linear motors 51 into translational movement along the z axis for pad-holders 1g, 1d, 2g and 2d is ensured by y-axis rotation of first lever 531 and of second lever 532. More specifically, the y-axis rotation of first lever 531 makes it possible to convert the x-axis translational movement of linear motors 51 into a z-axis translational movement for pad-holders 1g and 2g, while the y-axis rotation of second lever 532 makes it possible to convert the x-axis translational movement of linear motors 51 into a z-axis translational movement for pad-holders 1d and 2d.

The y-axis rotation of first lever 531 is achieved by a y-axis first pivot 5313 situated at the angle of the L of first lever 531. Actuation of linear motor 51 causes x-axis translation of third carriage 52 along rail 521. The x-axis translation of third carriage 52 causes the y-axis rotation of first lever 531 by pushing the first end of first lever 531 via first sliding pivot 5311. The y-axis rotation of first lever 531 drives a z-axis translational movement of the second end of first lever 531. The z-axis translation of the second end of first lever 531 drives a translation of support S1 via second sliding pivot 5312.

The y-axis rotation of second lever 532 is achieved by a second pivot 5323 of the y axis situated at the angle of the L of second lever 532. Actuation of linear motor 51 causes the x-axis translation of third carriage 52 along rail 521. The x-axis translation of third carriage 52 causes the y-axis rotation of second lever 532 by pushing the first end of second lever 532 via third sliding pivot 5321. The y-axis rotation of second lever 532 drives a z-axis translational movement of the second end of second lever 532. The z-axis translation of the second end of second lever 532 drives a translation of support S2 via fourth sliding pivot 5322.

The z-axis translational movement of pairs of pad-holders 1 and 2 makes it possible to impart a rotational movement to the manipulated medical instrument, by a movement of the same amplitude in opposite directions, of pad-holders 1g, 1d, 2g and 2d of a same pair (this principle is described in document WO2016/198800, also incorporated by reference). Thus, a single linear motor 51 may be used for each pair of pad-holders 1, 2, thereby simplifying the structure of the robot and limiting its mass and bulkiness. First lever 531 and second lever 532 are arranged in opposite directions such that translational movement of carriage 52 along the y axis causes translational movement, in the opposite direction and of the same amplitude, of second ends of first lever 531 and of second lever 532.

FIG. 5 schematically represents a perspective view of a sliding ball joint from the same example of a robotic platform integrating the inner mechanism of a robotic catheter module for translation and rotation of an elongate flexible medical instrument, according to one embodiment of the invention.

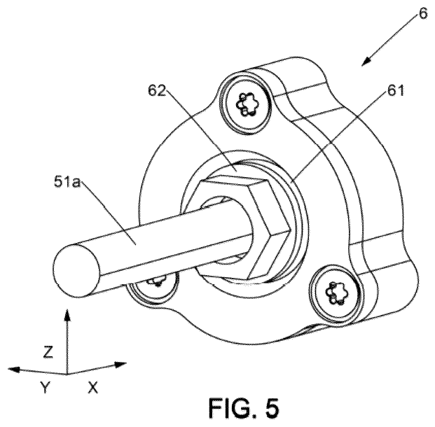

FIG. 6 schematically represents a front view of a sliding ball joint from the same example of a robotic platform integrating the inner mechanism of a robotic catheter module for translation and rotation of an elongate flexible medical instrument, according to one embodiment of the invention.

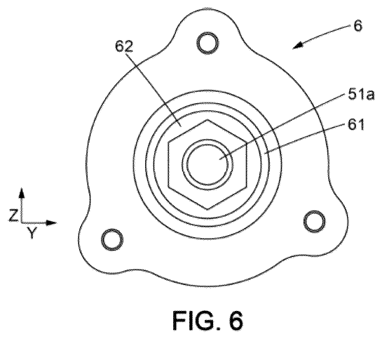

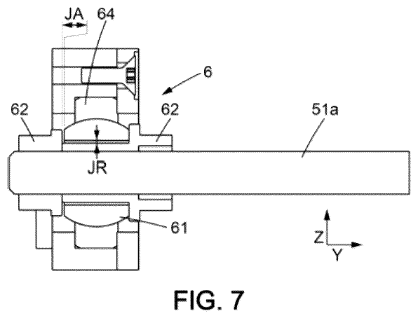

FIG. 7 schematically represents a cross-sectional view of a sliding ball joint from the same example of a robotic platform integrating the inner mechanism of a robotic catheter module for translation and rotation of an elongate flexible medical instrument, according to one embodiment of the invention.

FIGS. 5 to 7 represent detailed views of a sliding ball joint 6 for adjusting axial misalignment and radial misalignment, installed on output shaft 51a of linear motors 51. Sliding ball joint 6 installed on output shafts 41a of linear motors 41 is similar to sliding ball joint 6 installed on output shafts 51a of linear motors 51.

Sliding ball joint 6 comprises a ball joint 61 mounted about output shaft 51a of linear motor 51. Ball joint 61 is mounted on output shaft 51a so as to rotate freely about output shaft 51a. In the embodiment illustrated in FIGS. 5 to 7, ball joint 61 is mounted about a bearing 62 which is mounted about output shaft 51a, ball joint 61 being mounted to rotate freely about said bearing 62. In order to ensure the attachment of ball joint 61, bearing 62 comprises a shoulder in order to form a first stop at a first end of ball joint 61, and a nut 63 is screwed around output shaft 51a at the second end of ball joint 61 in order to form a second stop opposite to said first stop. A ring 64 is mounted about ball joint 61 and is adapted to slide around about said ball joint 61 in order to ensure the three rotational degrees of freedom of sliding ball joint 6, and thereby to compensate for an axial misalignment relative to output shaft 51a during assembly. In order to adjust for radial misalignment, ball joint 61 is, on the one hand, mounted about bearing 62 with a radial clearance JR, for example, a radial clearance of between 0.2 mm and 1 mm, and on the other hand, is mounted between the stops with an axial clearance JA, for example also between 0.2 mm and 1 mm. Radial clearance JR allows compensating for radial misalignment, and axial clearance JA allows ball joint 61 to slide radially in order to adjust for radial misalignment. The value of radial clearance JR may be adapted according to the maximum acceptable radial misalignment. The value of axial clearance JA may be adapted according to the maximum acceptable axial misalignment.

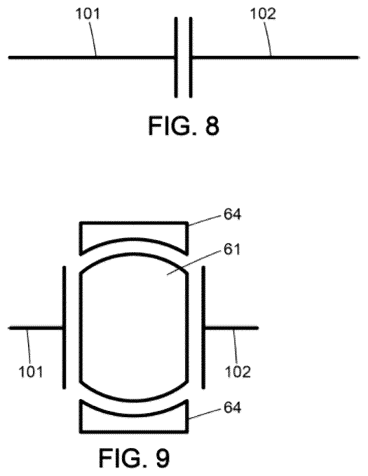

FIG. 8 schematically represents a first relative position between two shafts in a robotic platform.

Two shafts 101 and 102 are perfectly aligned with each other, i.e. their axes are exactly within the extension of one another.

FIG. 9 schematically represents a first configuration of a sliding ball joint, corresponding to the first relative position between two shafts in a robotic platform corresponding to FIG. 8. The sliding ball joint comprises a convex ball joint 61 positioned in a concave ring 64, ball joint 61 able to rotate freely within ring 64.

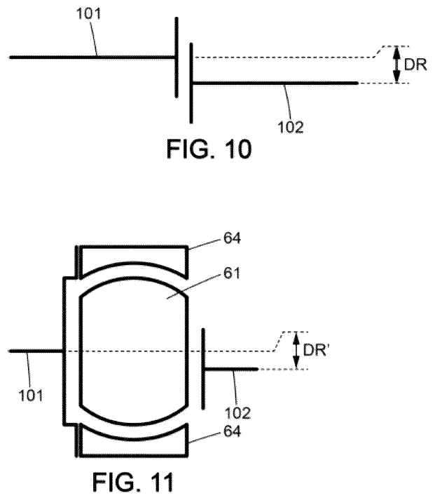

FIG. 10 schematically represents a second relative position between two shafts in a robotic platform.

The two shafts 101 and 102 are not perfectly collinear, i.e. their axes have a radial misalignment DR between them.

FIG. 11 schematically represents a second configuration of a sliding ball joint, corresponding to the second relative position between two shafts in a robotic platform corresponding to FIG. 10.

The use of a sliding ball joint comprising ball joint 61 positioned in ring 64, ball joint 61 able to rotate freely within ring 64, makes it possible to solve the problem of radial misalignment DR shown in FIG. 10, by means of radial offset DR′ between ball joint 61 on the one hand and second shaft 102 on the other hand.

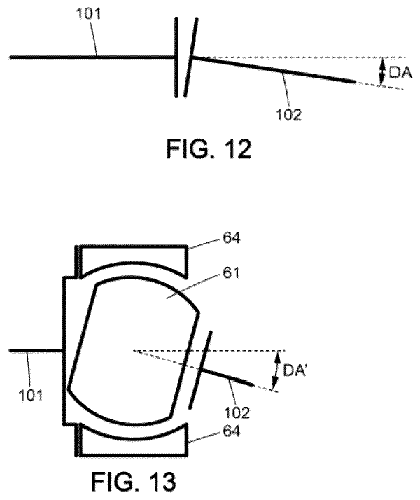

FIG. 12 schematically represents a third relative position between two shafts in a robotic platform.

The two shafts 101 and 102 are not perfectly aligned between each other, i.e. their axes have an axial misalignment DA between them.

FIG. 13 schematically represents a third configuration of a sliding ball joint, corresponding to the third relative position between two shafts in a robotic platform corresponding to FIG. 12.

The use of a sliding ball joint comprising ball joint 61 positioned in ring 64, ball joint 61 able to rotate freely within ring 64, makes it possible to resolve the problem of axial misalignment DA shown in FIG. 12, by means of axial offset DA′ between ball joint 61 on the one hand and ring 64 on the other hand.

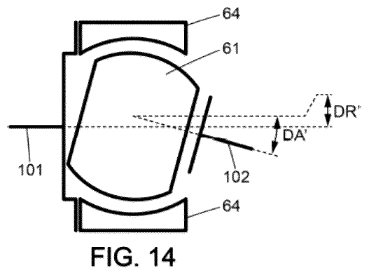

FIG. 14 schematically represents a fourth configuration of a sliding ball joint, corresponding to a combination of the second and third relative positions between two shafts in a robotic platform.

The use of a sliding ball joint comprising ball joint 61 positioned in ring 64, ball joint 61 able to rotate freely within ring 64, makes it possible to resolve both:
the problem of radial misalignment DR, by means of radial offset DR′ between ball joint 61 on the one hand and second shaft 102 on the other hand,
and the problem of axial misalignment DA, by means of axial offset DA′ between ball joint 61 on the one hand and ring 64 on the other hand.

Of course, the present invention is not limited to the examples and embodiment described and represented, but is likely to have numerous variations accessible to a person skilled in the art.

1-10. (canceled)

11. A robotic catheter module for translation and rotation of an elongate flexible medical instrument, comprising:
a support (S) having a longitudinal direction (y), a transverse direction (x) which is orthogonal to the longitudinal direction (y), and a vertical direction (z) which is orthogonal to the longitudinal direction (y) and to the transverse direction (x);
at least one pair of movable pads arranged face to face, adapted to be able to:
effect a longitudinal translation of the elongate flexible medical instrument, by gripping the elongate flexible medical instrument between the pads and by moving the pads together in translation in the longitudinal direction;
effect a rotation of the elongate flexible medical instrument about the longitudinal direction, by gripping the elongate flexible medical instrument between the pads and by moving the pads together in translation in the vertical direction, said pads moving in translation in opposite directions;
a device for driving the pads, comprising:
a first drive member for driving pads in the longitudinal direction (y), comprising a first actuator which is connected to the pads via at least one rolling-bearing slide connection;
a second drive member for driving pads in the vertical direction (z), comprising a second actuator which is connected to the pads via at least one rolling-bearing slide connection;
a third drive member for driving pads at least relative to one another in the transverse direction (x), comprising a third actuator which is connected to at least one pad via at least one rolling-bearing slide connection;
these three drive members for the pads being independent of one another such that the actuation of an actuator of one of the drive members does not displace the actuators of the two other drive members.

12. The robotic catheter module according to claim 11, wherein one among the first drive member, the second drive member, and the third drive member comprises a sliding coupling that is connected to the actuator of said drive member by a first shaft and that is connected to at least one pad by a second shaft, the sliding coupling allowing a translational movement of the second shaft relative to the first shaft along a plane perpendicular to the first shaft.

13. The robotic catheter module according to claim 12, wherein the sliding coupling via roller bearing comprises several balls, said balls being respectively housed in several housings supported by a same support, and able to roll on a same plane of a same part.

14. The robotic catheter module according to claim 13, wherein the sliding coupling comprises at least three balls.

15. The robotic module according to claim 12, wherein the sliding coupling is installed on the second shaft of the third drive member.

16. The robotic catheter according to claim 11, wherein the second actuator is a linear actuator and the second drive member comprises a conversion device that converts a translational movement of the second actuator into a translational movement of the pads along the vertical axis (z) by means of an intermediate adapter.

17. The robotic catheter module according to claim 16, wherein the intermediate adapter has an L shape and the axis of rotation of said intermediate adapter is located at the intersection of the two arms of the L, a first arm of the L being connected to a pad, the second drive member being connected to a second arm of the L, the first arm of the L preferably being shorter than the second arm of the L or else the first arm of the L preferably being at least 2 times or at least 3 times shorter than the second arm of the L.

18. The robotic catheter module according to claim 16, wherein the intermediate adapter comprises a sliding pivot connection at each of its two ends.

19. The robotic catheter module according to claim 11, wherein at least one among the first drive member, the second drive member, and the third drive member comprises a sliding ball joint for adjusting radial misalignment (DR) and for adjusting axial misalignment (DA), mounted about an output shaft so as to slide radially about said shaft.

20. The robotic catheter module according to claim 11, wherein: the first drive member comprises a single first actuator, the second drive member comprises a single second actuator, and the third drive member comprises a single third actuator.
